(19) United States
(12) Patent Application Publication
(10) Pub. No.: US 2016/0061816 A1
(43) Pub. Date: Mar. 3, 2016

(54) SCREENING METHOD FOR ION CHANNEL MODULATORS USING MUTATED BKCA CHANNEL

(71) Applicant: GWANGJU INSTITUTE OF SCIENCE AND TECHNOLOGY, Gwangju (KR)

(72) Inventors: Chul-Seung PARK, Gwangju (KR); Byoung-Cheol LEE, Gwangju (KR); Hyun-Ju KIM, Gwangju (KR)

(73) Assignee: GWANGJU INSTITUTE OF SCIENCE AND TECHNOLOGY, Gwangju (KR)

(21) Appl. No.: 14/783,568

(22) PCT Filed: Dec. 10, 2013
(86) PCT No.: PCT/KR2013/011414

§ 371 Date: Oct. 9, 2015

(30) Foreign Application Priority Data
Apr. 10, 2013 (KR) 10-2013-0039319

(51) Int. Cl. G01N 33/50

(52) U.S. Cl. CPC G01N 33/5023; G01N 2333/705

(57) ABSTRACT

The present disclosure relates to a doubly mutated BK_{Ca} channel construct and a novel cell-based screening system for ion channel modulators using the same. A doubly mutated BK_{Ca} channel-comprising cell-based system of the present invention, when compared to a control group, shows remarkably increased fluorescence caused by membrane depolarization, regardless of whether there is a separate increase of [Ca²⁺]ᵢ, and shows activity (movement in the negative direction of a G/V curve) that is further triggered by a known activator (for example, CTBIC). Therefore, the system of the present disclosure is capable of more effectively and accurately analyzing the activity of an ion channel, and thus can be effectively applied not only to the separation/identification of ion channel modulators but also to screening for a therapeutic agent for a condition, disease or disorder related to the modulation of a BK_{Ca} channel.

### TECHNICAL FIELD

The present invention was developed with government support under project number “NN09570” awarded by the Ministry of Education, Science and Technology of Korea. The managing department name for the project is National Research Foundation of Korea (NRF), the project title is “National Leading Research (Strategic Research) Laboratory Grant Program”, the project task title is “Research for Derivation of a Novel Ion Channel Protein for Targeting Urinary Incontinence and Functional Control Thereof”, the project was performed by the Gwangju Institute of Science and Technology, and the total project period was from Sep. 1, 2012 to Aug. 31, 2013.

This application claims the benefit of Korean Patent Application No. 10-2013-0039319, filed on Apr. 10, 2013, which is incorporated herein by reference in its entirety.

The present invention relates to a doubly mutated BK_{Ca} channel construct and a novel cell-based screening system for ion channel modulators using the same.

### BACKGROUND ART

Membrane depolarization and increase in intracellular Ca²⁺ concentration activate large (or big)-conductance calcium-activated potassium (BK_{Ca}) channels known as BK or Maxi-K channels (Salkoff, et al., 2006; Cui, et al., 2009). These channels play significant physiological roles in neuronal excitability, neurotransmitter release, contraction of smooth muscle cells and frequency tuning of hair cells (Brenner, et al., 2000; Nelson, et al., 1995; Fettiplace and Fuchs, 1999). BK_{Ca} channels are composed of a pore-forming α-subunit and a modulatory β-subunit. The α-subunit of BK_{Ca} channels is composed of seven transmembrane domains (Catterall, 1995), wherein the C-terminal includes two modulators capable of modulating K⁺ conductance (RCK) domains, which form a gating ring in response to intercellular Ca^{2|} concentration (Jiang, et al., 2001).

BK_{Ca} channels are considered attractive therapeutic targets, since they are closely related with hypertension, coronary artery spasm, urinary incontinence and a number of neurological disorders (Ghatta, et al., 2006). BK_{Ca} channel-deficient mice exhibit symptoms such as urinary incontinence, bladder overactivity and erectile dysfunction (Meredith, et al., 2004; Werner, et al., 2005). Further, BK_{Ca} channel dysfunction may cause cerebellar ataxia and paroxysmal movement disorders (Lee and Cui, 2010). Activation of BK_{Ca} channels stabilizes cells by increasing efflux of K⁺, which leads to hyperpolarization. Accordingly, activators of BK_{Ca} channels can confer therapeutic advantages, such as decrease in cellular excitability and relaxation of smooth muscle cells.

Up to now, it is understood that patch clamping analysis is the most reliable technique for developing ion channel modulators. However, manual patch clamping demonstrates very low throughput and requires a high level of technological expertise. Accordingly, there have been developed a lot of alternative methods with higher throughput, such as flux assays, radioligand binding assays, fluorescence-based assays and automated patch clamping methods (Zheng, et al., 2004). Nevertheless, it was hard to establish cell-based assays for analyzing BK_{Ca} channels, which meet the requirements for the methods with a higher throughput.

Throughout the entire specification, many articles and patent documents are cited and referred to herein. The cited articles and patent documents are incorporated herein by reference in their entirety, and thus the technological level of the present invention and context of the present invention are more clearly explained.

### DISCLOSURE

### Technical Problem

The inventors of the present invention have made an effort to develop an effective cell-based high-throughput screening system for identification of and research on ion channel modulators. As a result, the present inventors have prepared a hyperactive BK_{Ca} channel in which amino acids at positions G733 and N736 in a wild type BK_{Ca} channel gene are mutated, and identified that the BK_{Ca} channel is activated by a voltage pulse at a low Ca^{2|} concentration and potentiated by a BK_{Ca} channel activator (for example, CTBIC) on a cell-based assay platform, thereby accomplishing the present invention.

Therefore, it is an object of the present invention to provide a method for screening ion channel modulators.

It is another object of the present invention to provide a mutated BK_{Ca} channel protein.

It is a further object of the present invention to provide a recombinant vector including a nucleotide sequence encoding an amino acid sequence shown in SEQ ID. NO: 4.

It is yet another object of the present invention to provide a cell transformed by the recombinant vector.

It is yet another object of the present invention to provide a method for screening a therapeutic agent for diseases, disorders or conditions related to the modulation of a BK_{Ca} channel.

Other objects and advantages of the present invention will be clearly described through the following detailed description, the claims and drawings.

### Technical Solution

In accordance with one aspect of the present invention, the present invention provides a method for screening ion channel modulators including:

(a) treating a cell including a nucleotide sequence encoding an amino acid sequence in which amino acids at positions G733 and N736 in a wild type BK_{Ca} channel gene are mutated with a test material; and

(b) analyzing the activity of the mutated BK_{Ca} channel in the cell, wherein the test material is determined to be an ion channel activator when the test material potentiates the activity of the mutated BK_{Ca} channel, and the test material is determined to be an ion channel inhibitor when the test material inhibits the activity of the mutated BK_{Ca} channel.

In accordance with another aspect of the present invention, the present invention provides a mutated BK_{Ca} channel protein comprised of an amino acid sequence shown in SEQ ID. NO: 4.

In accordance with a further aspect of the present invention, the present invention provides a recombinant vector including (a) a nucleotide sequence encoding an amino acid sequence shown in SEQ ID NO: 4; (b) a promoter operatively linked to the nucleotide sequence; and (c) a terminator.

In accordance with yet another aspect of the present invention, the present invention provides a cell transformed with the recombinant vector.

The inventors of the present invention endeavored to develop an effective cell-based high-throughput screening system for identification and research of ion channel modulators. As a result, the present inventors have prepared a hyperactive BK_{Ca} channel in which amino acids at positions G733 and N736 in a wild type BK_{Ca} channel gene are mutated, and found that the BK_{Ca} channel is activated by a voltage pulse at a low Ca²⁺ concentration and potentiated by a BK_{Ca} channel activator (for example, CTBIC) on a cell-based assay platform.

Cells are electrically stabilized through activation of various K⁻ ion channels with increase in potassium concentration as an intracellular secondary transmitter. The intracellular potassium concentration temporarily rises by the extracellular influx (for example, voltage-dependent potassium channel) or the release from intracellular repository [for example, ER (endoplasmic reticulum)], thereby activating K⁺ ion channels. K⁺ ion channels may be divided into big conductance calcium-activated potassium channels (BK_{Ca}), intermediate conductance calcium-activated potassium channels (IK_{Ca}) and small conductance calcium-activated potassium channels (SK_{Ca}), depending on the amount of channel permeating K⁺ ions per unit hour.

BK_{Ca} channels are characterized by large conductance of K⁺ ions permeating cell membranes and are also called Maxi-K or slo1. BK_{Ca} channels are activated (opened) by changes in membrane potential and/or by increase in concentration of intracellular calcium ions ([Ca²⁺]ᵢ). Opening of BK channels allows intracellular K⁺ ions to flow through from the cell, causing changes in electrochemical concentration. This results in cell membrane hyperpolarization (an increase in the electrical potential across the cell membrane) and a decrease in cell excitability (a decrease in the probability that the cell will transmit an action potential).

BK_{Ca} channels play a critical role in the regulation of various physiological processes including contraction of smooth muscle, neuronal excitability, electrical tuning of cochlea hair cells, and the like. BK_{Ca} channels are composed of a pore-forming α-subunit and a modulatory β-subunit. More particularly, the α-subunit of BK_{Ca} channels is composed of a unique transmembrane domain (S0), voltage sensing domains (S1-S4), K⁺ channel pore domains (S5 and S6), and a cytoplasmic C-terminal domain (CTD) (contains binding sites for Ca²⁺, called “calcium bowls”, within the second RCK domain) consisting of a pair of RCK domains.

Patch clamping methods have been typically employed in order to identify and investigate ion channel modulators, but have a major drawback of very low efficiency. In order to overcome this drawback, various alternative methods (for example, automated patch clamping methods, flux assays, fluorescence-based assays, and the like) have been proposed and implemented. However, there is still a need for development of more effective and convenient channel analysis methods.

The present invention provides a novel method for screening cell-based ion channel modulators. Further, the method of the present invention has a merit in that changes in ion channel activation can be detected accurately in a very simple manner through a commercially available fluorescence assay method.

First, the method according to the present invention prepares a doubly mutated BK_{Ca} channel (G733D/N736K) (pre-(a) step).

According to one embodiment of the invention, a recombinant vector to be used in preparation of the doubly mutated BK_{Ca} channel (G733D/N736K) according to the present invention is a recombinant vector including (a) a nucleotide sequence encoding an amino acid sequence shown in SEQ ID NO: 4; (b) a promoter operatively linked to the nucleotide sequence; and (c) a terminator. More specifically, the recombinant vector of the present invention includes a recombinant vector including (i) a nucleotide sequence shown in SEQ ID NO: 3; (ii) a promoter operatively linked to the nucleotide sequence in (i) and being active in an animal cell and ensuring the formation of an RNA molecule; and (iii) a 3′-non-coding region being active in an animal cell and causing 3′-end polyadenylation of the RNA molecule. The nucleotide sequence and amino acid sequence for a wild type BK_{Ca} channel are shown in SEQ ID NO: 1 and SEQ ID NO: 2, respectively.

As used herein, the term “promoter” refers to a DNA sequence regulating expression of a coding sequence or functional RNA. In the recombinant vector of the present invention, a target nucleotide sequence is operatively linked to the promoter. As used herein, the term “operatively linked” refers to a functional linkage between a nucleic acid expression regulatory sequence (for example: a promoter sequence, a signal sequence, or a transcription modulator binding site array) and other nucleic acid sequences. The regulatory sequence is capable of regulating transcription and/or translation of other nucleic acid sequences.

The vector system according to the present invention can be constructed by various methods known in the art, details of which are disclosed in Sambrook et al., Molecular Cloning, A Laboratory Manual, Cold Spring Harbor Laboratory Press (2001), which is incorporated herein by reference.

In the case where the recombinant vector of the present invention is applied to eukaryotic cells (for example, AD-293 cells), promoters capable of regulating transcription of a nucleotide sequence encoding an amino acid sequence shown in SEQ ID NO: 4 of the present invention may be utilized. Such promoters may include a promoter derived from a mammalian virus, a promoter derived from a genome of a mammalian cell and a promoter derived from a yeast cell. Examples of the promoters may include CMV (cytomegalovirus) promoter, adenovirus late promoter, vaccinia virus 7.5K promoter, SV40 promoter, HSV tk promoter, RSV promoter, EF1 alpha promoter, metallothionein promoter, beta-actin promoter, human IL-2 gene promoter, human IFN gene promoter, human IL-4 gene promoter, human lymphotoxin gene promoter, human GM-CSF gene promoter, yeast (S. cerevisiae) GAPDH (Glyceraldehyde 3-phosphate dehydrogenase) promoter, yeast (S. cerevisiae) GAL1 to GAL10 promoter and yeast (Pichia pastoris) AOX1 or AOX2 promoter, without being limited thereto. More specifically, the promoter is CMV promoter.

Further, the expression construct used in the present invention includes a polyadenylation sequence (for example, bovine growth hormone terminator (BGH pA) and SV40-derived polyadenylation sequence).

Furthermore, the vector according to the present invention further includes a selection marker. According to one embodiment of the invention, the vector of the present invention includes an antibiotic resistance gene typically used in the art, for example, a gene imparting resistance to neomycin, geneticin, ampicillin, kanamycin, hygromycin, streptomycin, penicillin, chloramphenicol, gentamycin, carbenicillin or tetracycline, without being limited thereto.

Methods for introducing the vector of the present invention to a host cell may employ various methods known in the art. For example, when a host cell is a prokaryotic cell, the introduction of the vector of the present invention into a host cell can be carried out according to a CaCl₂ method (Cohen, et al., Proc. Natl. Acac. Sci. USA, 69: 2110-2114(1972)), Hanahan's method (Hanahan, D., J. Mol. Biol., 166: 557-580(1983)), an electroporation method (Dower, et al., Nucleic. Acids Res., 16: 6127-6145(1988)), and the like. When the host cell is a eukaryotic cell, the vector of the present invention can be introduced into a host cell through lipofection, electroporation, liposome-mediated transformation (Wong, et al., Gene, 10: 87-94(1980)) and retrovirus-mediated transformation [Chen, H. Y., et al., (1990), J. Reprod. Fert. 41:173-182; Kopchick, J. J. et al., (1991) Methods for the introduction of recombinant DNA into chicken embryos. In Transgenic Animals, ed. N. L. First & F. P. Haseltine, pp. 275-293, Boston; Butterworth-Heinemann; Lee, M.-R. and Shuman, R. (1990) Proc. 4th World Congr. Genet. Appl. Livestock Prod. 16, 107-110)], microinjection, particle bombardment, yeast spheroplast/cell fusion used in YAC, agrobacterium-mediated transformation used in a plant cell, and the like. More specifically, the introduction of the vector of the present invention is performed by a lipofection method.

Next, a test material is brought into contact with a cell transformed with the recombinant vector of the present invention. Cells including the nucleotide sequence of the present invention are not particularly limited. Specifically, the cells include AD-293 cells. The term “test material” used in the screening method of the present invention refers to an unknown material to be used in screening in order to examine whether or not the material affects the activity of a doubly mutated BK_{Ca} channel protein. The test material includes chemical compounds, antisense oligonucleotides, siRNA (small interference RNA), shRNA (small hairpin RNA or short hairpin RNA), miRNA (microRNA), peptides and natural extracts, without being limited thereto.

When the test material to be used in the screening method of the present invention is a chemical material, the material may be a single compound or a mixture of compounds (for example, cells or tissue cultures). The test material may be obtained from libraries of synthetic or natural compounds. The methods for obtaining libraries of such compounds are known in the art. Synthetic compound libraries are commercially available from Maybridge Chemical Co. (UK), Comgenex (USA), Brandon Associates (USA), Microsource (USA) and Sigma-Aldrich (USA). Libraries of natural compounds are commercially available from Pan Laboratories (USA) and MycoSearch (USA). The test materials may be obtained by various combination library methods known in the art, for example, biological libraries, spatially addressable parallel solid phase or solution phase libraries, synthetic library methods requiring deconvolution, a “one-bead, one-compound” library method, and synthetic library methods using affinity chromatography selection. The methods for synthesizing molecule libraries are disclosed in DeWitt, et al., Proc. Natl. Acad. Sci. U.S.A. 90, 6909, 1993; Erb, et al., Proc. Natl. Acad. Sci. U.S.A. 91, 11422, 1994; Zuckermann, et al., J. Med. Chem. 37, 2678, 1994; Cho et al., Science 261, 1303, 1993; Carell, et al., Angew. Chem. Int. Ed. Engl. 33, 2059, 1994; Carell, et al., Angew. Chem. Int. Ed. Engl. 33, 2061; Gallop, et al., J. Med. Chem. 37, 1233, 1994, and the like.

As used herein, the term “antisense oligonucleotides” refers to strands of DNA or RNA containing a nucleic acid sequence complementary to a specific mRNA or derivatives thereof. Antisense oligonucleotides prevent protein translation of mRNA by binding to a complementary sequence in mRNA strands. The term “complementary” as used herein means that antisense oligonucleotides are complementary sufficiently to be capable of selectively hybridizing to a target (for example, genes affecting the activity of BK_{Ca} channel proteins) under given hybridization or annealing conditions, preferably physiological conditions. Complementary antisense oligonucleotides may have one or more mismatched bases, and are refer tp both substantially complementary and perfectly complementary oligonucleotides. More specifically, antisense oligonucleotides are perfectly complementary oligonucleotides. Antisense oligonucleotides are 6 to 100 nucleotide base pairs in length, more particularly, 8 to 60 nucleotide base pairs in length, most particularly 10 to 40 nucleotide base pairs in length.

As used herein, the term “siRNA” refers to a nucleic acid molecule that can mediate RNA interference or gene silencing (see: WO 00/44895, WO 01/36646, WO 99/32619, WO 01/29058, WO 99/07409 and WO 00/44914). Since siRNA can inhibit expression of a target gene, siRNA can be provided as an efficient gene knockdown method or a gene therapy method. siRNAs were first discovered in plants, insects, drosophila and parasites. Recently, siRNAs have been developed/utilized in the study of mammalian cells (Degot S, et al., 2002; Degot S, et al., 2004; Ballut L, et al., 2005).

siRNA molecules capable of being employed in the present invention may have a double stranded structure consisting of a sense strand (for example, a sequence corresponding to a gene mRNA sequence affecting the activity of BK_{Ca} channel proteins) and an antisense strand (for example, a sequence complementary to a gene mRNA sequence affecting the activity of BK_{Ca} channel proteins), which are located opposite each other. Further, siRNA molecules capable of being employed in the present invention may have a single stranded structure consisting of a self-complementary sense strand and an antisense strand.

siRNAs are not limited to perfectly complementary double stranded RNA, but include portions that are not base paired due to mismatches (corresponding bases are not complementary), bulges (having no corresponding bases at one side of strands), and the like. Specifically, siRNAs are 10 to 100 base pairs in length, more specifically 15 to 80 base pairs in length, and far more specifically 20 to 70 base pairs in length.

As used herein, the term “shRNA (small hairpin RNA or short hairpin RNA)” refers to an RNA sequence forming a tight hairpin turn that can be used to silence target gene expression via RNA interference. A vector for introduction into a cell may be employed in the delivery of shRNA, and mostly a U6 promoter capable of expressing shRNAs may be employed. The use of such a vector allows shRNAs to always be delivered to daughter cells, thereby ensuring inheritance of gene silencing. The shRNA hairpin structure is cleaved by cellular machinery into siRNA, which is then bound to the RNA-induced silencing complex (RISC). This complex binds to and cleaves mRNAs which match the siRNA that is bound thereto. shRNAs are transcribed by RNA polymerase III. The production of shRNA in mammalian cells may cause an interferon response as cells recognize shRNAs as a virus attack and seek a means of protection. In addition, shRNAs may be employed in plants and other systems, and do not necessarily require the U6 promoter. In the case of plants, a CaMV (cauliflower mosaic virus) 35S promoter which is a typical promoter containing a very strong expression ability may be employed.

As used herein, the term “microRNA (miRNA)” refers to a single stranded RNA molecule of 21-25 nucleotides in length, which regulates gene expression of eukaryotes through binding to 3′-UTR of mRNA (messenger RNA) (Bartel DP, et al., Cell, 23;116(2): 281-297(2004)). miRNA is first transcribed as a primary transcript, which is processed into a stem-loop structured pre-miRNA by Drosha (RNaseIII type enzyme), and then further processed into a mature miRNA in the cytoplasm with cleavage action by Dicer [Kim V N, et al., Nat Rev Mol Cell Biol., 6(5): 376-385(2005)]. Thus prepared miRNA regulates expression of a target protein and thus is involved in generation, proliferation and apoptosis of cells, lipid metabolism, tumor formation, and the like [Wienholds E, et al., Science, 309(5732): 310-311(2005); Nelson P, et al., Trends Biochem. Sci., 28: 534-540(2003); Lee R C, et al., Cell, 75: 843-854(1993); and Esquela-Kerscher A, et al., Nat Rev Cancer, 6: 259-269(2006)].

As used herein, the term “peptide” refers to a linear molecule of amino acid residues linked by peptide bonds. The peptides of the present invention may be prepared by chemical synthesis methods known in the art, specifically by solid-phase synthesis techniques (Merrifield, J. Amer. Chem. Soc. 85: 2149-54(1963); Stewart, et al., Solid Phase Peptide Synthesis, 2nd. ed., Pierce Chem. Co.: Rockford, 111(1984)).

Finally, the degree of activation of BK_{Ca} channel proteins in a cell treated with a test material is analyzed. The activity measurement may be easily performed through fluorescence determination as described in below. As a result, the test material is determined to be an ion channel activator when the test material potentiates the activation of mutated BK_{Ca} channels while the test material is determined to be an ion channel inhibitor (blocker) when the test material inhibits the activation of mutated BK_{Ca} channels.

As used herein, the term “ion channel activator” refers to a material that potentiates the activation (opening) of mutated BK_{Ca} channels. As used herein, the term “ion channel inhibitor” refers to a material that inhibits activation (opening) of mutated BK_{Ca} channels.

Since the mutated BK_{Ca} channels of the present invention are hyperactive, the mutated BK_{Ca} channels have a sufficient activity under a low concentration of [Ca²⁺]ᵢ as compared to wild type BK_{Ca} channels, which allows experimental data to be more clearly analyzed.

According to one embodiment of the invention, the analysis of the mutated BK_{Ca} channel activity of the present invention is performed through fluorescence measurement for T1⁺ ion concentrations. The fluorescence measurement for T1⁺ ion concentrations may be carried out in a simple and convenient way by a standard fluorometer using commercially available assay methods known in the art (for example, FluxOR™).

According to one embodiment of the invention, the mutated BK_{Ca} channel of the present invention may be activated by membrane depolarization regardless of [Ca²⁺]ᵢ.

According to one embodiment of the invention, the membrane depolarization is induced stepwise by applying voltage pulses from −80 mV to 200 mV with increments of 10 mV.

According to one embodiment of the invention, the conductance-voltage relationship (G-V) of the mutated BK_{Ca} channel of the present invention shifts toward the negative voltage direction.

According to one embodiment of the invention, the activity of the BK_{Ca} channel of the present invention exhibits an ˜2.3 times increase in fluorescence signals by stimulus with 10 μM of CTBIC (4-chloro-7-(trifluoromethyl)-10H-benzofuro[3,2-b]indole-1-carboxylic acid) (see: FIG. 5c).

In according with yet another embodiment of the present invention, the present invention provides a method for screening a therapeutic agent for BK_{Ca} channel activity-associated diseases, disorders or conditions, including:

(a) treating a cell including a nucleotide sequence encoding an amino acid sequence in which amino acids at positions G733 and N736 in a wild type BK_{Ca} channel gene are mutated with a test material; and

(b) analyzing the activity of the mutated BK_{Ca} channel in the cell, wherein the test material is determined to be an ion channel activator when the test material potentiates the activity of the mutated BK_{Ca} channel, and the test material is determined to be an ion channel inhibitor when the test material inhibits the activity of the mutated BK_{Ca} channel.

The method according to the present invention includes, as an effective ingredient, a cell transformed with the mutated BK_{Ca} channel of the present invention, and therefore the related illustration is omitted here for the sake of conciseness.

According to one embodiment of the invention, diseases, disorders or conditions associated with modulation of the BK_{Ca} channel of the present invention include cardiovascular diseases, obstructive or inflammatory airway diseases, lower urinary tract disorders, erectile-dysfunction, anxiety and anxiety-related conditions, epilepsy and pain.

As used herein, the term “cardiovascular diseases” refers to a general term that describe various conditions affecting the heart, heart valves, blood and vasculature, and therefore includes diseases affecting the heart or blood vessels. According to one embodiment of the invention, examples of cardiovascular diseases may include atherosclerosis, atherothrombosis, coronary artery disease, ischemia, reperfusion injury, hypertension, restenosis, arteritis, myocardial ischemia or ischemic heart disease, stable and unstable angina, stroke, congestive heart failure, aortic diseases such as aortic coarctation or aortic aneurysm and peripheral vascular diseases. As used herein, the “peripheral vascular diseases (PVDs)” refers to diseases of the blood vessels located outside the heart and central nervous system, which are often encountered in stenosis of the extremities. For example, PVDs may be classified as functional PVDs occurring due to stimuli such as cold, emotional stress or smoking without defects in vessels, and organic PVDs occurring due to physical defects in vessel systems such as atherosclerosis, partial inflammation or traumatic injury.

According to one embodiment of the invention, examples of obstructive or inflammatory airway diseases may include hyperactive airway response, pneumoconiosis, aluminosis, anthracosis, asbestosis, lithosis, ptilosis, siderosis, silicosis, tobacco toxicosis, byssinosis, sarcoidosis, berylliosis, emphysema, acute respiratory distress syndrome (ARDS), acute lung injury (ALI), acute or chronic infectious pulmonary disease, chronic obstructive pulmonary disease (COPD), bronchitis, chronic bronchitis, wheezy bronchitis, hyperactive airway response or aggravated cystic fibrosis, or cough including chronic cough, aggravated hyperactive airway response, pulmonary fibrosis, pulmonary hypertension, inflammatory pulmonary disease, and acute or chronic respiratory infection disease.

As used herein, the term “lower urinary tract disorders” refers to all of the lower urinary tract disorders characterized by overactive bladder having or without having leaking urine, urinary frequency, urgency to urinate, and nocturia. Therefore, lower urinary tract disorders in the present invention may include urinary bladder symptoms such as overactive bladder, overactive detrusor muscle, unstable bladder, detrusor hyperreflexia, sensory urgency to urinate and detrusor overactivity; lower urinary tract disorder symptoms including urinary incontinence or urge incontinence, urinary stress incontinence, slow urination, terminal dribbling, anuria and/or obstructive voiding symptom requiring allowable pressure to squeeze urine out; and irritating symptoms such as urinary frequency and/or urge to urinate. Further, examples of lower urinary tract disorders may include neurogenic bladder resulting from neurological injury including stroke, Parkinson's disease, diabetes, multiple sclerosis, peripheral neuropathy, or spinal cord injury, without being limited thereto. Further, examples of lower urinary tract disorders may include prostatitis, interstitial cystitis, prostatic hyperplasia, and spastic bladder in spinal cord injury patients. According to one embodiment of the invention, examples of lower urinary tract disorders may include overactive bladder, unstable bladder, overactive detrusor muscle, detrusor instability, detrusor hyperreflexia, sensory urge to urinate, urinary incontinence, urinary urge incontinence, urinary stress incontinence, neurogenic (reflex) urinary incontinence, slow urination, terminal dribbling, dysuria and spastic bladder, without being limited thereto.

As used herein, the term “erectile dysfunction” refers to sexual dysfunction characterized by the inability to develop or maintain an erection of the penis during sexual activity, which is closely related with endothelial cell dysfunction.

According to one embodiment of the invention, diseases, disorders or conditions related to modulation of BK_{Ca} channels of the present invention may include pain disorders; anxiety and anxiety-related conditions such as generalized anxiety disorders, panic disorder, obsessive compulsive disorder, social phobia, performance anxiety, posttraumatic stress disorder, acute stress reaction, adjustment disorder, hypochondria, separation anxiety disorder, agoraphobia and specific phobias; and epilepsy such as generalized seizure including simple partial seizure, complex partial seizure, secondary generalized seizure, absence seizure, myoclonic seizure, clonic seizure, tonic seizure, tonic clonic seizure and atonic seizure, without being limited thereto.

In addition, examples of specific phobia-related anxieties may include any kind of anxiety disorder that amounts to a fear related to exposure to not only animals, insects, thunderstorms, driving, flying, height or crossing bridges, small confined or narrow spaces, water, blood or injury but also injection or surgical treatment and dental procedures, without being limited thereto.

Furthermore, pain disorders are disorders accompanying pains. Examples of pain disorders may include acute pains such as musculoskeletal pains, postoperative pain, and surgical pain; chronic pains such as chronic inflammatory pains (for example, rheumatoid arthritis and osteoarthritis), neuropathic pains (for example, post herpetic neuralgia, trigeminal neuralgia and sympathetically maintained pains) and cancer-related pains and fibromyalgia; migraine-related pains; (both chronic and acute) pains, and/or fever and/or infection such as rheumatic fever; symptoms related with other viral infections such as influenza or common cold; lower back pains and neck pains; headache; toothache; sprains and strains; myositis; neuralgia; synovitis; arthritis including rheumatoid arthritis; degenerative joint diseases including osteoarthritis; gout and ankylosing spondylitis; tendinitis; bursitis; skin-related conditions such as psoriasis, eczema, burns and dermatitis; sports injuries; and injuries caused by surgery and dental procedures, without being limited thereto.

### Advantageous Effects

Features and advantages of the present invention are summarized below:

(a) The present invention relates to a doubly mutated BK_{Ca} channel construct and a novel cell-based screening system for ion channel modulators using the same.

(b) The doubly mutated BK_{Ca} channel-comprising cell-based system of the present invention, when compared to a control group, shows remarkably increased fluorescence caused by membrane depolarization, regardless of whether there is a separate increase of [Ca²]ᵢ.

(c) Further, the doubly mutated BK_{Ca} channel-comprising cell-based system of the present invention shows potentiated activity triggered by a known activator (for example, CTBIC) (G/V curves shift in the negative direction).

(d) Therefore, compared to conventional methods (for example, an automated patch clamping method), the system of the present invention is capable of more effectively and far more accurately analyzing the activity of an ion channel, and thus can be effectively applied not only to the separation/identification of ion channel modulators but also to screening of therapeutic agents for diseases, disorders or conditions related to the modulation of a BK_{Ca} channel.

The effects of the present invention are not limited to the above-mentioned effects, and other effects not mentioned herein may be clearly understood by a person skilled in the art from the disclosure set forth below.

### DESCRIPTION OF DRAWINGS

FIG. 1 shows the preparation and features of hyperactive mutated BK_{Ca} channels. FIG. 1a is a schematic view of the α-subunit of human BK_{Ca} channels used in the present invention. Mutations at positions G733D and N736K in the RCK2 domain are indicated in blue. FIG. 1b shows locations of mutated positions in an interfacial crystalline structure (Protein Data Bank ID: 3NAF) between the RCK1 (yellow) domain and RCK2 (violet) domain. FIG. 1c shows representative macroscopic current recording results of cells transiently transfected with WT (wild type) or G733D/N736K BK_{Ca} channel constructs.

FIG. 2 is a result showing identification of stable cell lines expressing WT or G733D/N736K BK_{Ca} channels. FIG. 2a is an immunoblot analysis result for stable cell lines expressing WT or G733D/N736K BK_{Ca} channels. A control group (Mock) was transfected with a pcDNA3.1 empty vector construct. 30 μg of cell lysate was loaded in each lane. Membranes were reacted with an anti-BK_{Ca} channel antibody or an anti-GAPDH antibody as a control group. FIG. 2b shows representative macroscopic current recording results for WT and G733D/N736K BK_{Ca} channels which are stably expressed in the presence of 100 nM [Ca²⁺]ᵢ. The ionic currents were evoked with voltage steps of 100 ms to test potentials ranging from −80 mV to 100 mV with increments of 10 mV. The holding voltage was −100 mV. FIG. 2c shows normalized G-V relationships for steady-state currents of WT (open circles) and G733D/N736K (filled circles) BK_{Ca} channels. The membrane was held at −100 mV and was then increased stepwise from −80 mV to 200 mV in increments of 10 mV. Channel currents were recorded in the presence of 100 nM [Ca²⁺]ᵢ. Conductance values were obtained from peak tail currents, and normalized for maximum conductance observed in the absence of CTBIC. Data points were fitted using the Boltzmann function.

FIG. 3 shows electrical physiological properties for WT and mutated BK_{Ca} channels. FIG. 3a shows effects of WT and G733D/N736K BK_{Ca} channels on G-V relationships, depending on changes in [Ca²⁺]ᵢ. The concentrations of [Ca²⁺]ᵢ were set to 0 μM (squares), 0.1 μM (circles), 1 μM (triangles) or 10 μM (inverted triangles). The membrane was held at −100 mV and was then increased stepwise from −80 mV to 200 mV in increments of 10 mV. Conductance values were obtained from peak tail currents, and normalized for maximum conductance. Data points were fitted using the Boltzmann function. FIG. 3b shows half-activation voltages (V_{1/2}) of WT and G733D/N736K BK_{Ca} channels at different intracellular concentrations of Ca²⁺. Each data point represents mean values obtained from five experiments±standard error of the mean (S.E.M). FIG. 3c shows resting membrane potentials (RMP) of steady-state cell lines expressing WT (open square) or G733D/N736K (filled square) BK_{Ca} channels. Each data point represents mean values obtained from 33 experiments±S.E.M. Indication: **, p<0.001 according to paired Student's t-test.

FIG. 4 shows potentiation of the activity of WT and G733D/N736K channels by a BK_{Ca} channel activator. FIGS. 4a and 4b show representative views of macroscopic current recordings for WT (FIG. 4a) and G733D/N736K (FIG. 4b) BK_{Ca} channels in the presence or absence of 10 μM CTBIC. [Ca²⁺]ᵢ was fixed at 100 nM, and CTBIC was applied to an intracellular side of the membrane. The ionic currents were increased with voltage steps of 100 ms to test potentials ranging from −80 mV to 100 mV in increments of 10 mV. The holding voltage was −100 mV. FIG. 4c shows normalized G-V relationships for steady-state currents of WT and G733D/N736K BK_{Ca} channels. Prior to recording, a vehicle as a control group (square) or 10 μM CTBIC (circle) was applied on a membrane patch. The membrane was held at −100 mV and was then increased stepwise from −80 mV to 200 mV in increments of 10 mV. FIG. 4d shows changes in half-activation voltages (V_{1/2}) of WT and G733D/N736K BK_{Ca} channels induced by 10 μM CTBIC. Each data point represents mean values obtained from five experiments±S.E.M. Indication: **, p<0.001 according to paired Student's t-test.

FIGS. 5a-5c show analysis results for suitability of steady-state cell lines expressing G733D/N736K BK_{Ca} channels in high throughput screening using a fluorescence-based assay platform. Fluorescence signals for parental AD-239 cells (FIG. 5a), and fluorescence signals for cells stably expressing WT (FIG. 5b) or G733D/N736K (FIG. 5c) BK_{Ca} channels were measured. In order to monitor the activity of BK_{Ca} channels, the parental AD-239 cells, WT cells and G733D/N736K cells were loaded with FluxOR™ dye. Prior to reading the fluorescence signals, cells were pre-treated with a BK_{Ca} channel activator (10 μM CTBIC; filled symbols) or a vehicle (DMSO; open symbols) for 30 minutes. Basal fluorescence was measured for two minutes, followed by treating cells with a stimulus buffer containing 10 mM free K⁺, thereby depolarizing cell membranes. The fluorescence signals were detected by using a hybrid multi-mode microplate reader, Synergy™ H1. Symbols: 0 μM CTBIC, open symbols; and 10 μM CTBIC, filled symbols.

### MODE FOR INVENTION

Hereinafter, the present invention will be described in more detail with reference to the following examples. However, it will be apparent to those skilled in the art that the present invention is not limited to these examples and that various modifications, substitutions, changes, and equivalents thereof can be made without departing from the scope of the invention.

### EXAMPLE

### Experimental Materials and Experimental Methods

### Mutagenesis and Construction of Stable Cell Lines

A wild type (WT) human BK_{Ca} channel-coding region (GenBank accession number, NM002247 and NP 002238.2) was subcloned into a pcDNA3.1(+) mammalian expression vector (Invitrogen, Carlsbad, Calif.). A mutated BK_{Ca} channel (G733D/N736K) was obtained by means of site-directed mutagenesis of a WT plasmid using QuikChange Site-Directed Mutagenesis Kit (Stratagene, Santa Clara, Calif.).

Derivatives of HEK293 cell line, i.e., AD-293 cells (Stratagene) were maintained in DMEM (Dulbecco's Modified Eagle's Medium; Thermo, Waltham, Mass.) supplemented with 10% FBS (fetal bovine serum; Thermo) and antibiotics. The cells were cultured at 37° C. under a constant humidity 5% CO₂ environment. In order to isolate stable cell lines, pcDNA3.1 vectors containing a WT BK _{Ca} channel construct or a G733D/N736K mutated construct were transfected into AD-293 cells by using Polyfect reagent (Qiagen, Valencia, Calif.) in accordance with the manufacturer's instructions. Cells were cultured in a medium containing 1 mg/ml of geneticin (Gibco-RRL, Carlsbad, Calif.), and the medium was replaced with fresh medium every two days.

### Immunoblot Analysis

Cells were subjected to lysis using a buffer including 20 mM HEPES (pH 7.5; Sigma), 120 mM NaCl (Sigma), 5 mM EDTA (Sigma), 1% Triton X-100 (Sigma), 0.5 mM DTT (dithiothreitol; Sigma), 1 mM PMSF (phenylmethylsulfonyl fluoride; Sigma) and Protease Inhibitor Cocktail (Roche Applied Science, Indianapolis, Ind.). After storing specimens on ice for 30 minutes, the lysates were subjected to centrifugation at 12,500 rpm for 25 minutes in order to precipitate insoluble materials. After adding 5× SDS gel loading buffer (250 mM Tris-Cl (pH 6.8), 500 mM DTT, 10% SDS (Biorad), 0.5% bromophenol blue (Sigma) and 50% glycerol (USB product)), the resulting mixture was reacted at 37° C. for 15 minutes. Proteins were subjected to electrophoresis and blotted on a PVDF membrane (GE Healthcare Life Sciences). The membrane was blocked by stirring at room temperature for one hour using 1× TBS-T (1× Tris-buffered saline with Tween-20) including 3% BSA, followed by washing with 1× TBS-T three times, then reacting at room temperature in 10 ml 1× TBS-T including a primary antibody (anti-BK_{Ca} antibody, 1:250 dilution; BD Biosciences, San Jose, Calif.); or an anti-GAPDH antibody, 1:5000 dilution; Young In Frontier, Seoul, Korea)) overnight. Subsequently, the membrane was washed with 1× TBS-T three times, followed by reacting with 5 ml 1× TBS-T including a secondary antibody (1:10,000 dilution; Jackson ImmunoResearch, West Grove, Pa.) for 45 minutes. Finally, the membrane was washed with 1× TBS-T three times, which was blocked with ECL western blotting detection reagent (Amersham Biosciences, Little Chalfont, Buckinghamshire, UK). The blots were wrapped in plastic wrap and then exposed onto X-ray film (Konica, Tokyo, Japan).

### Electrophysiological Recordings and Data Analysis

Macroscopic current recordings were performed using a gigaohm seal patch clamp method. Patch pipettes were made from borosilicate glass (WPI, Sarasota, Fla.), and then fire polished with resistance of 3-5 MΩ. The channel currents were amplified using Axopatch 200B amplifier (Axon Instruments, Foster City, Calif.), low-pass filtered at 1 or 2 kHz using a four-pole Bessel filter, and then digitized at a rate of 10 or 20 points/ms using a Digidata 1200 A digitizer (Axon Instruments). The ionic currents of BK_{Ca} channels were activated by voltage-clamped pulses delivered from a holding potential of −100 mV to membrane potentials ranging from −80 mV to 200 mV in increments of 10 mV. The intracellular and extracellular solutions contained, unless otherwise specified, 116 mM KOH (Sigma), 4 mM KCl (Sigma), 10 mM HEPES and 5 mM EGTA (Sigma), and were titrated to pH 7.2 using MES (2-(N-morpholino)ethanesulfonic acid). In order to precisely measure [Ca²⁺]ᵢ, the appropriate amount of total Ca²⁻ to be added to the intracellular solution was calculated using MaxChelator software (Patton, et al., 2004; http://maxchelator.stanford.edu/).

In order to measure the resting membrane potential (RMP), the intracellular solution was adjusted to pH 7.2, and included 5 mM NaCl, 140 mM KCl (Sigma), 3 mM Mg-ATP (Sigma), 0.5 mM MgCl₂ (Sigma), 0.33 mM CaCl₂ (Sigma) and 1 mM EGTA. The extracellular solution was adjusted to pH 7.4, and included 145 mM NaCl, 4.5 mM KCl, 5 mM glucose (Sigma), 1.8 mM CaCl₂, 1 mM MgCl₂ and 5 mM HEPES. The pH values required for the intracellular and extracellular solutions were adjusted using NMDG and NaOH, respectively. The membrane potentials were measured one minute after conventional whole-cell configuration was obtained. Clampex 8.0 or 8.1 (Axon Instruments) and Origin 6.1 (OriginLab Corp., Northampton, Mass.) software packages were used for the acquisition and analysis of recording data.

### Fluorescence Measurement

Commercially available FluxOR™ Potassium Ion Channel Assay (Invitrogen) was used in order to perform a fluorescence-based assay for BK_{Ca} channels. AD-293 cells stably expressing WT and G733D/N736K BK_{Ca} channels were plated onto a poly-D-lysine (Sigma)-coated 96-well microplate (Corning) (5×10⁴ cells per well). Cells were pre-cultured with a test compound, CTBIC (4-chloro-7-(trifluoromethyl)-10H-benzofuro[3,2-b]indole-1-carboxylic acid) for 30 minutes in advance of the recording. The fluorescence signal was measured using a Synergy™ H1 Hybrid Multi-Mode Microplate Reader (BioTek Instrument, Inc., Winnoski, Vt.) with Gen5 software. The recording was also performed as a control group in AD-293 cells transfected with a pcDNA3.1 empty vector construct. FluxOR™ dye provides signals at excitation and emission wavelengths of 488 nm and 525 nm, respectively. The BK_{Ca} channel was stimulated by adding 100 mM free K⁺ to a culture medium. The microplate was read every 15 seconds in order to identify whether or not the system of the present invention is suitable for high throughput screening using a standard fluorimeter.

### Experimental Results

### Preparation of Hyperactive BK_{Ca} Channel Mutant and Identification of Properties Thereof

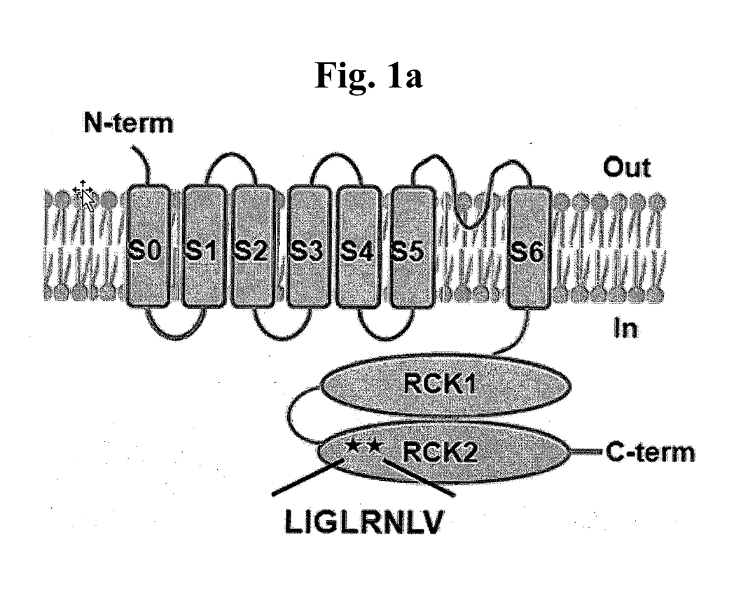

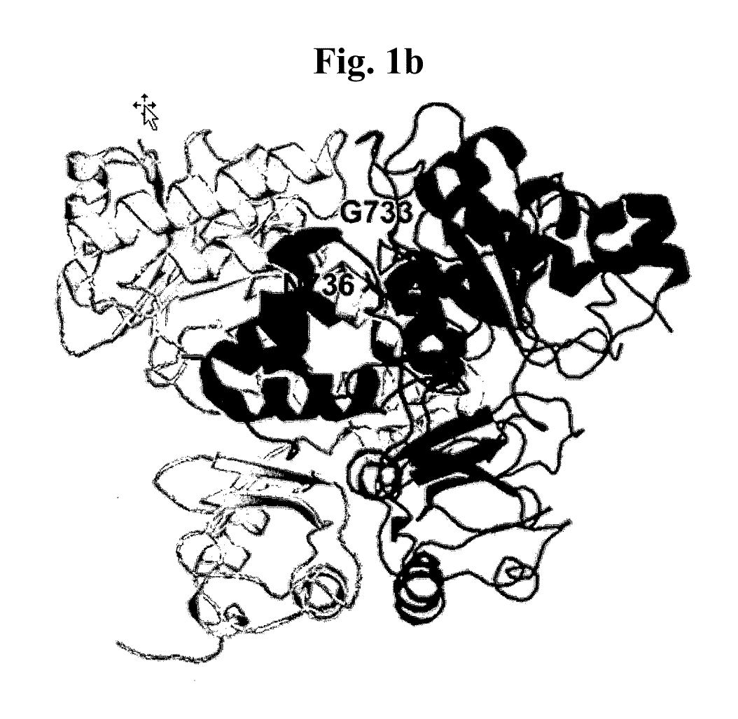

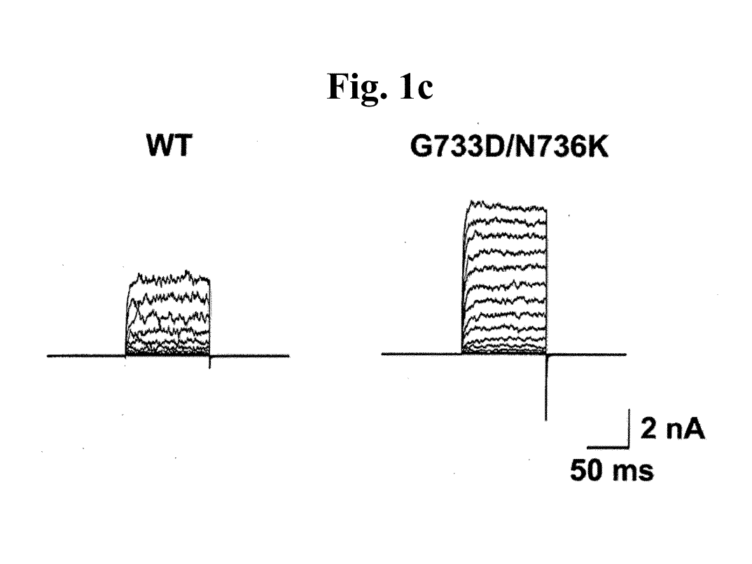

The present inventors had previously reported that BK_{Ca} channel activity is strongly affected by mutations in the flexible interface between the two RCK domains in the channel (Kim, et al., 2008). Mutations in the RCK2 domain shifted a voltage activation curve in the negative direction, thereby stabilizing the open conformation of channels, which lead to activation of BK_{Ca} channels (Kim, et al., 2008). In addition, a doubly mutated BK_{Ca} channel (G733D/N736K; FIG. 1a and FIG. 1b) including substitutions at two amino acids within the RCK2 domain was prepared and functional characteristics thereof were examined. The present inventors introduced an expression vector including WT human BK_{Ca} channel or human G733D/N736K mutated BK_{Ca} channel to AD-293 cells via transient transfection. In the absence of intracellular Ca²⁺, the mutated channel showed much lower membrane voltages as compared to WT BK_{Ca} channel (FIG. 1c).

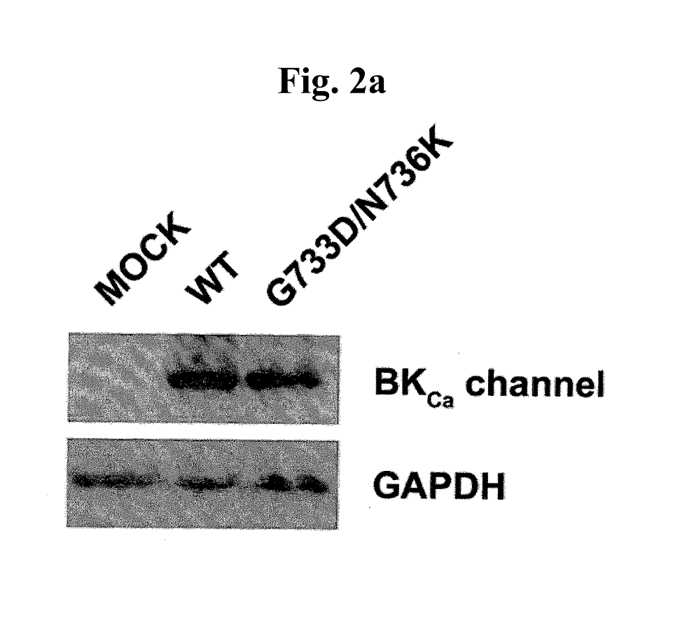

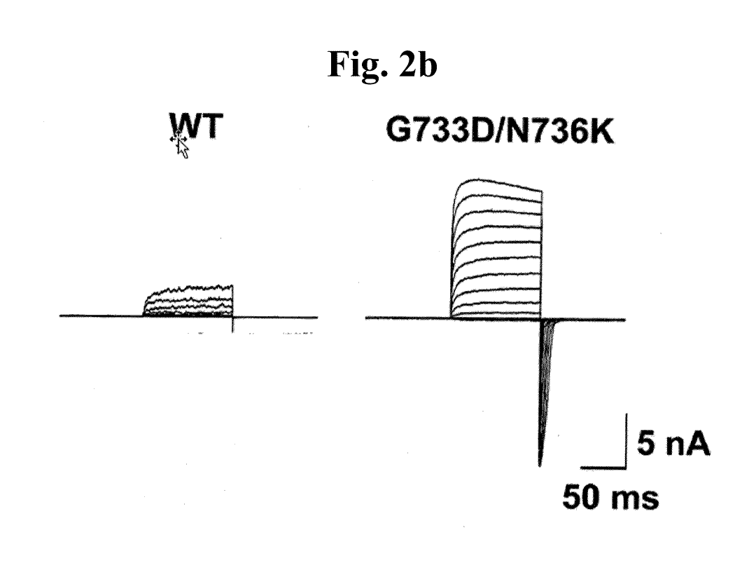

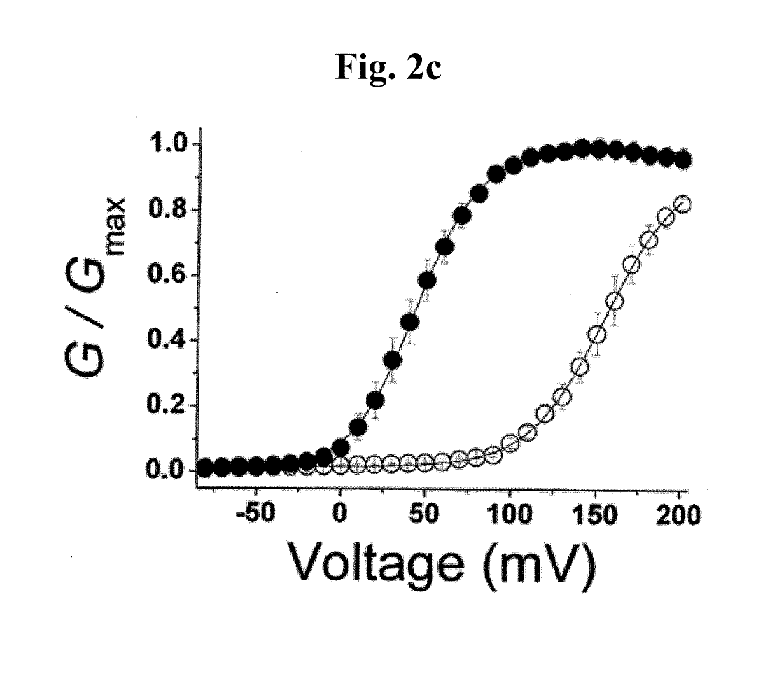

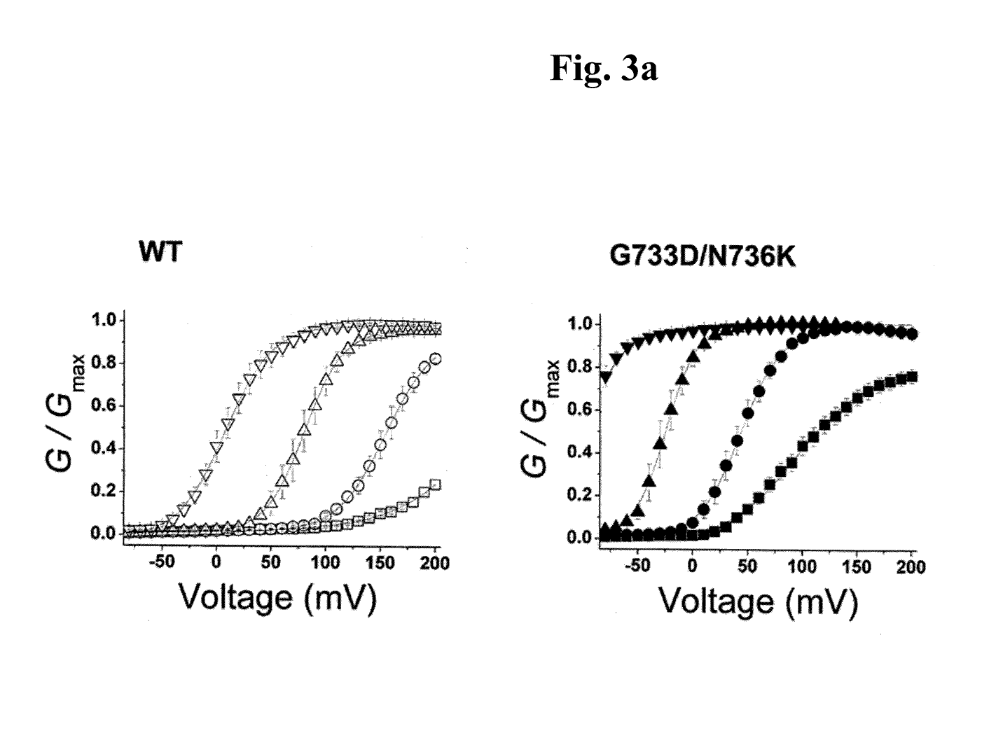

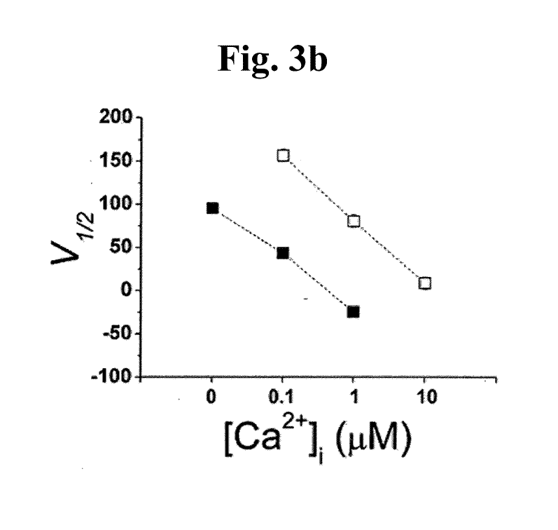

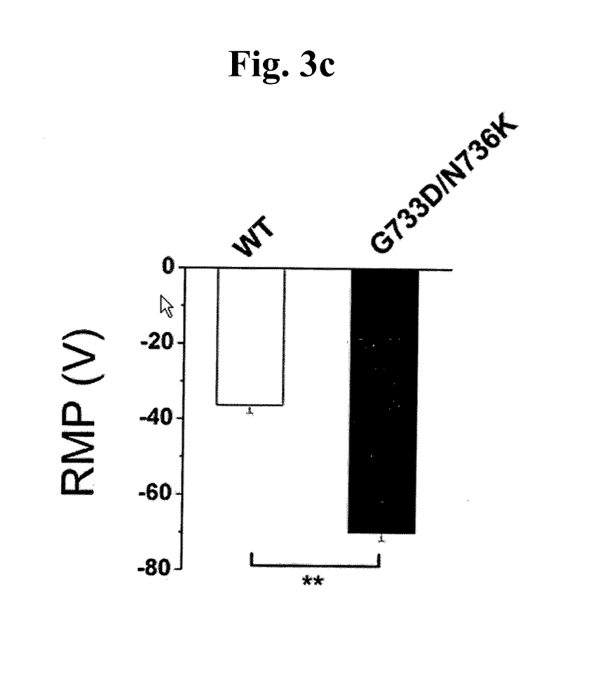

Subsequently, the transfected cells were selected in a medium including geneticin for 3 weeks to establish stable cell lines. Stable expression of the BK_{Ca} channel proteins was detected by immunoblot analysis using mouse anti-BK_{Ca} channel antibodies (FIG. 2a). No protein bands were observed in the parental cell line, but 130 kDa immunoactive bands were observed in the transfected cells (FIG. 2a), which correspond to the expected size for the BK_{Ca} channels. The expression levels for WT and G733D/N736K BK_{Ca} channel were comparable. The stably expressed WT and mutated BK_{Ca} channels were further examined by electrophysiological methods. The extracellular (bath, solution) and intracellular (pipette) solutions had the same concentration of K⁺ (120 mM), but various [Ca²⁺]ᵢ in each solution. Voltage pulses ranging from −80 mV to 200 mV were applied from a holding voltage of −100 mV in increments of 10 mV. In the case where the channels are activated by both membrane depolarization and 100 nM [Ca²⁺]ᵢ, WT and G733D/N736K channels triggered K⁺ current (FIG. 2b). G733D/N736K mutants remarkably shifted the conductance-voltage (G-V) relationships in the negative voltage direction (FIG. 2c). The half-activation voltage (V_{1/2}) of the G733D/N736K channel mutants exhibited about 110 mV of negative change from 100 nM [Ca²⁺]ᵢ. Furthermore, the ionic current of the channel mutants was completely activated by the voltage pulse in the absence of Ca²⁺ (FIG. 3a and FIG. 3b). Shift in V_{1/2} over a broad range of [Ca²]ᵢ was generated, which indicates that the double mutation caused changes in endothelial balance between closed states and open states of the BK_{Ca} channels (Kim, et al., 2008). These results mean that the G733D/N736K mutated channels stably expressed in AD-239 cell lines are capable of being activated by suitable depolarization of the membrane voltage at resting [Ca²⁺]ᵢ.

### Potentiation of G733D/N736K Channel Activity by Known BK_{Ca} Channel Activators

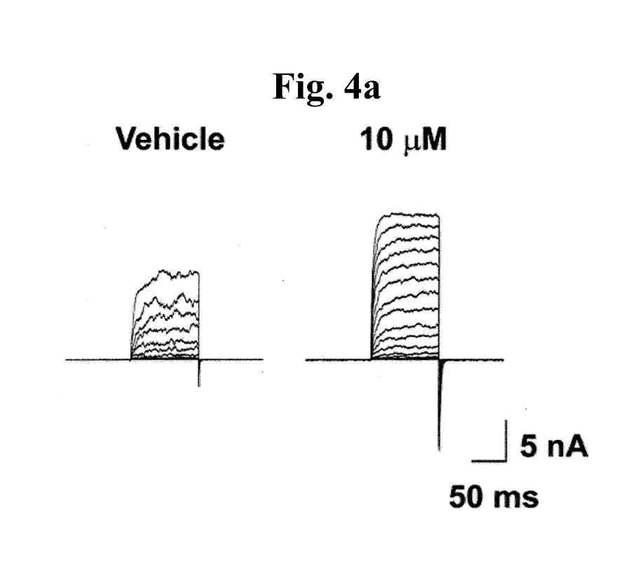

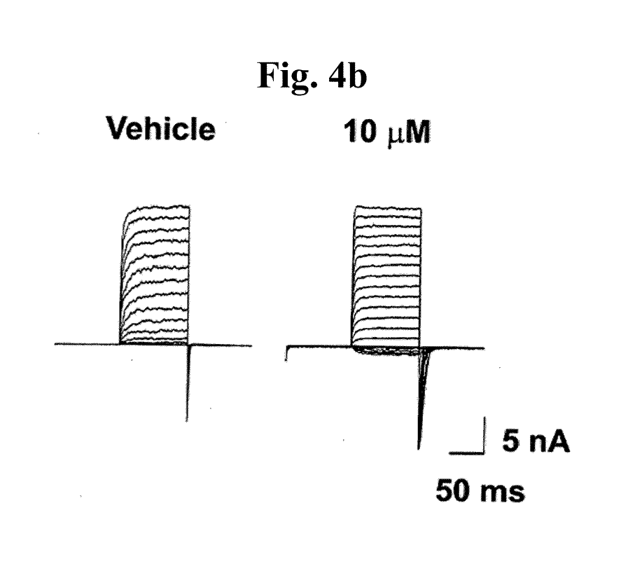

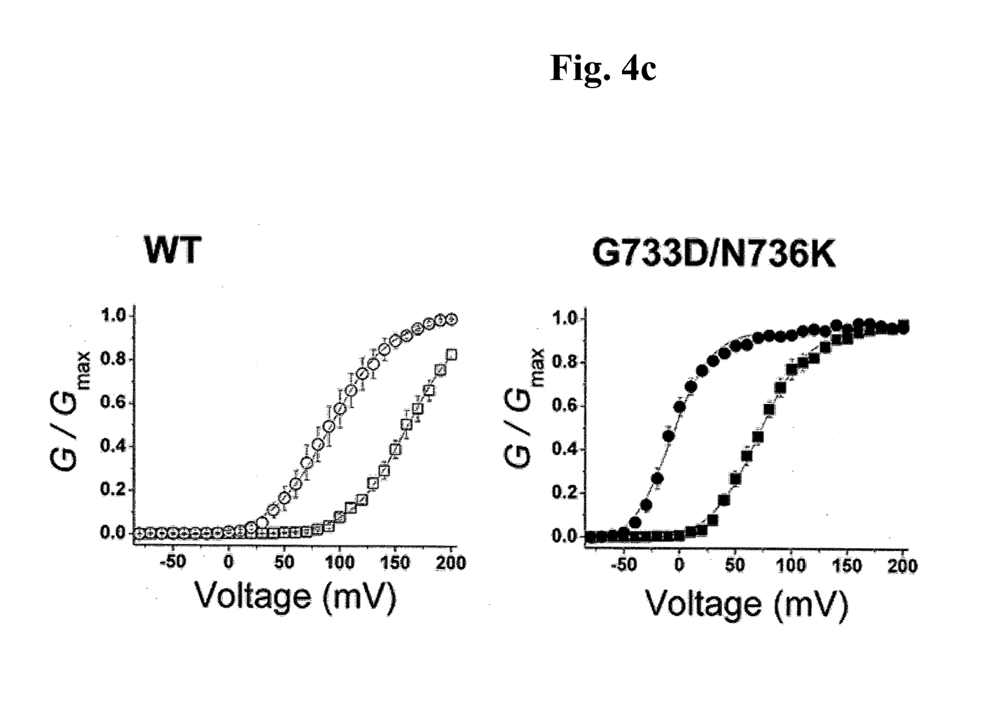

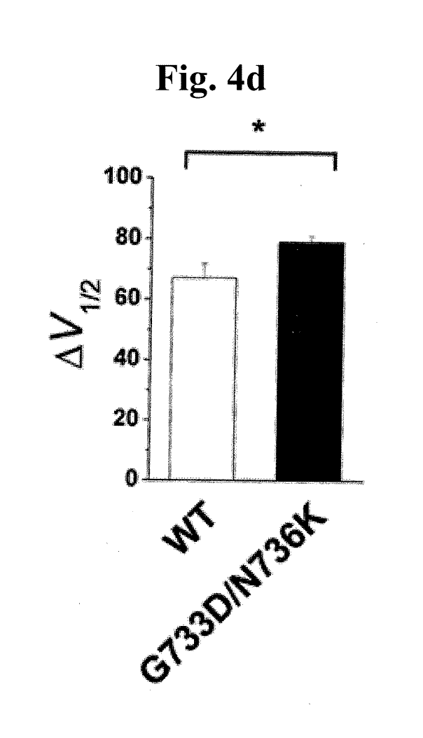

In order to evaluate the suitability of cell lines stably expressing G733D/N736K channels as a platform for identification of BK_{Ca} channel modulators, the function of a strong BK_{Ca} channel activator, CTBIC, which potentiates mutated channel activity, was investigated (Gormemis, et al., 2005; Lee, et al., 2012). The addition of 10 μM CTBIC to a membrane patch strongly potentiated ionic current in both WT BK_{Ca} channels (FIG. 4a) and G733D/N736K mutated BK_{Ca} channels (FIG. 4b). The relative conductance (G/Gₘₐₓ) was obtained by normalizing the tail currents (evoked by a step hyperpolarization to −100 mV from a given voltage) to the maximum tail current. The G/Gₘₐₓ values at 0 μM and 10 μM CTBIC were fitted using a Boltzmann function. The application of CTBIC allowed the G-V curves for WT and G733D/N736K channels to be shifted in the negative direction (FIG. 4c). The shift in the V_{1/2} value for the G733D/N736K channel (V_{1/2}^{free} vs. V_{1/2}^{10 μM}) was much larger than the shift for the WT channel (FIG. 4d).

### Strong Potentiation of G733D/N736K BK_{Ca} Channel on a Cell-Based Assay Platform

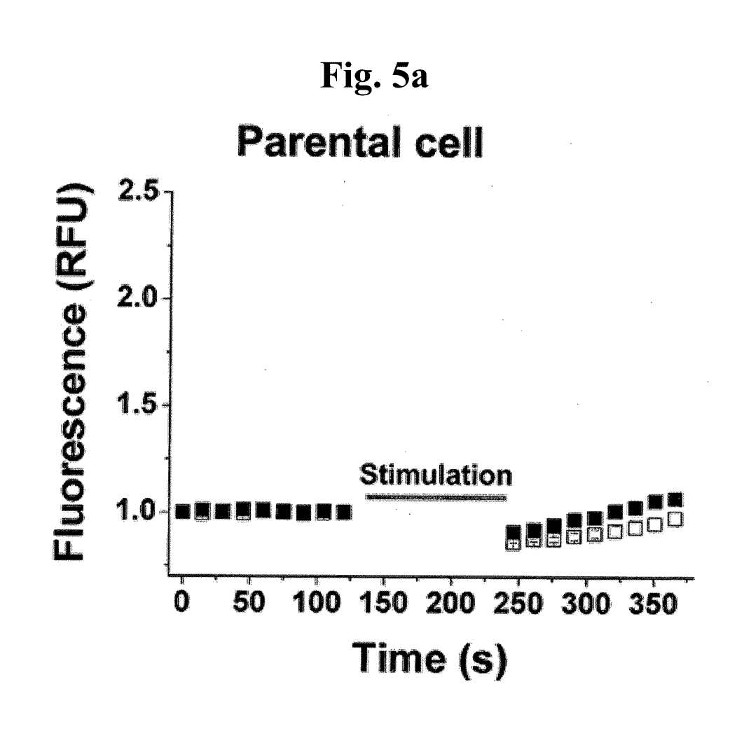

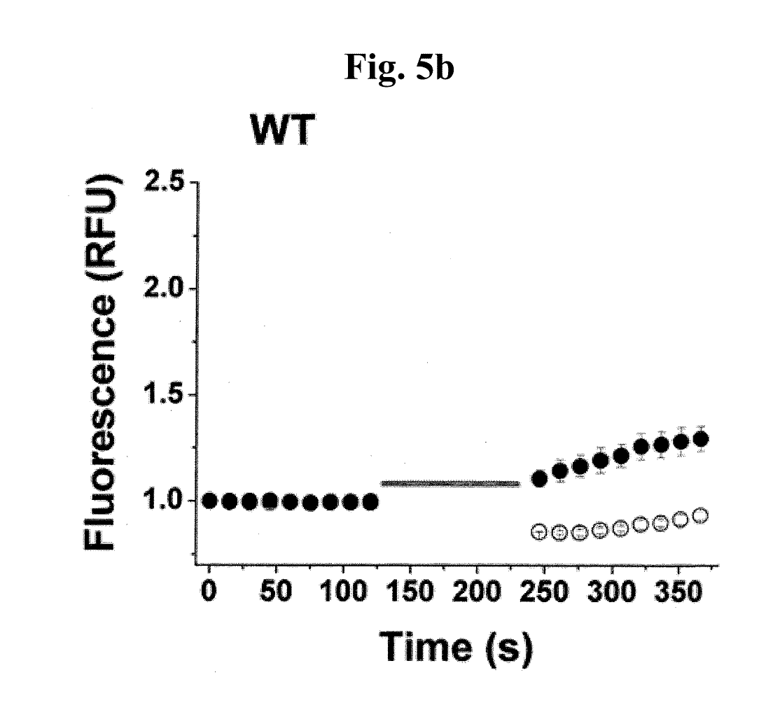

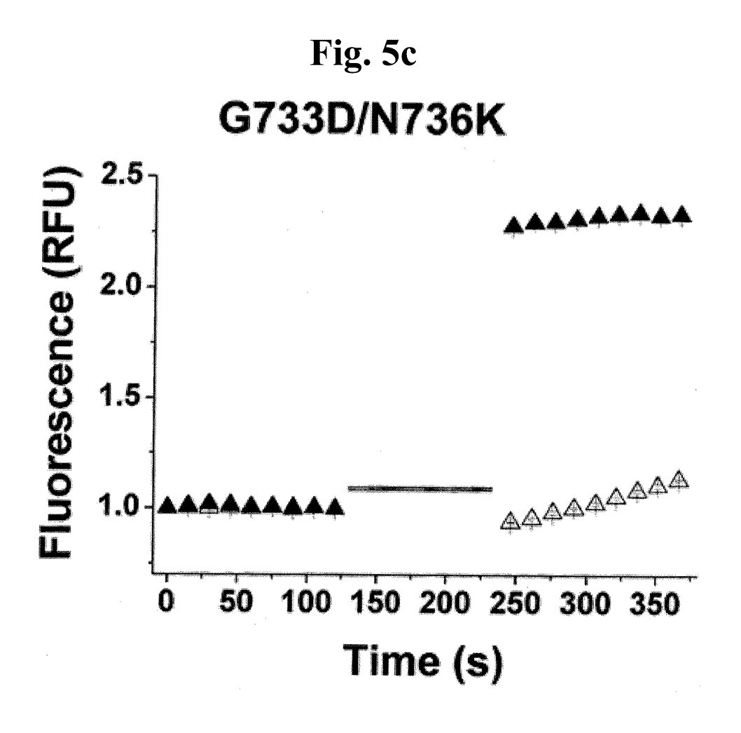

Finally, the present inventors performed experiments to determine whether cells stably expressing hyperactive BK_{Ca} channels are suitable for high-throughput screening for channel activators. Commercially available FluxOR™ dye was used as a general cell-based assay for K⁺ channels. Cell lines stably expressing WT or G733D/N736K BK_{Ca} channels were plated onto a poly-D-lysine-coated 96-well Plate and cultured. When cells reached 80% to 90% confluence, FluxOR™ signals were measured. Further, the fluorescence signals from parental AD-293 cell lines were measured as a control group. Prior to reading out the fluorescence, the cells were pre-treated with 10 μM CTBIC or DMSO (control group). Basal fluorescence was measured for two minutes, followed by treating cells with a stimulus buffer containing 10 mM free K⁺, thereby depolarizing cell membranes. The addition of 10 μM CTBIC demonstrated a minimum impact on the induced fluorescence increase in the parental cell line (FIG. 5a). In the case where stable cell lines expressing the WT channel were investigated, the present inventors could observe about 1.3-fold fluorescence increase by the addition of 10 μM CTBIC (FIG. 5b). However, stable cell lines expressing G733D/N736K channel mutants showed much greater response to the channel activator than cell lines expressing the WT channel (FIG. 5c). Relative fluorescence unit (RFU) values increased up to 2.3-fold as compared to basal responses. The above-mentioned results indicate that a platform using cells stably expressing G733D/N736K BK_{Ca} channels is capable of being used in high throughput screening for BK_{Ca} channel modulators.

### Additional Discussion

In spite of physiological importance and therapeutic power of BK_{Ca} channels, chemical modulators for BK_{Ca} channels have not been intensively investigated up to now. A major drawback related to screening for BK_{Ca} channel modulators is the absence of cell-based assay methods suitable for high throughput screening. Unlike other voltage-gated K⁺ channels, the activation of the BK_{Ca} channel at resting [Ca²⁻]ᵢ requires a big depolarization which is not generated under normal physiological conditions, whereas in the case of [Ca²⁻]ᵢ reaching micromole ranges, the activation is performed by slight depolarization. [Ca²⁺]ᵢ may be increased by activation of cell signaling pathways inducing the opening of intracellular Ca²⁺-permeable channels or the release of Ca²⁺ from the intracellular stores. The very elaborate sensitivity of BK_{Ca} channels to [Ca²⁺]_{I} necessitates an accurate modulation of sub-membrane Ca²⁺ concentration. For these reasons, there has been an enormous endeavor to modulate [Ca²⁺]ᵢ so as to investigate BK_{Ca} channel modulators: for instance, an improved automated patch clamping method including the application of compounds in order to modulate [Ca^{2|}]ᵢ has recently been reported (Ido, et al., 2012).

In this study, the present inventors developed a novel cell-based system to perform high throughput screening for BK_{Ca} channel modulators which can be utilized in fluorescence-based assays without requiring increased [Ca²⁺]ᵢ. Since prior studies suggested an important interaction between potential flexible interfaces between the two RCK domains in the BK_{Ca} channel (Kim, et al., 2008), the present inventors prepared hyperactive mutants including G733D and N736K mutants of the RCK2 domain, and identified characteristics thereof. In patch clamping analysis for cell lines stably expressing G733D/N736K BK_{Ca} channels, it was confirmed that the mutant channels were completely activated by applying voltages in the absence of intracellular Ca^{2|}, whereas the WT channels ware not activated. In addition, the present inventors identified that by employing commercially available assays for K⁺ channels, the fluorescence signals derived from G733D/N736K channels were much higher than the fluorescence signals derived from WT channels. As expected, the resting membrane potentials (RMP) of cell lines stably expressing G733D/N736K BK_{Ca} channels showed remarkably more negative voltages than RMP of cells expressing WT BK_{Ca} channels (FIG. 3c). More particularly, RMPs of cells expressing WT and G733D/N736K channels were −36.3±2.2 V and −70.0±2.1 V, respectively. The fact that cells expressing mutant channels had much lower negative RMP means than the mutant channels can mediate the opening of the release of K^{|} at resting [Ca^{2|}]ᵢ and membrane voltages, which further supports the idea that the mutated BK_{Ca} channels are hyperactive at resting conditions.

Conclusively, it is understood from the suggested results that stable cell lines expressing hyperactive BK_{Ca} channels are new cell-based assay systems to be used in the investigation of the BK_{Ca} channel activity. The above-mentioned cell lines are expected be very useful in screening novel activators for BK_{Ca} channels by using both fluorescence-based platforms and automated electrophysiological methods.

Although the present invention has been described with reference to some embodiments in conjunction with the accompanying drawings, it should be understood that the foregoing embodiments are provided for illustration only and are not to be construed in any way as limiting the present invention, and that various modifications, changes, alterations, and equivalent embodiments can be made by those skilled in the art without departing from the spirit and scope of the invention. Therefore, the true scope and spirit of the invention is indicated by the following claims and their equivalents.

### REFERENCES

Brenner, R., Perez, G. J., Bonev, A. D., Eckman, D. M., Kosek, J. C., Wiler, S. W., Patterson, A. J., Nelson, M. T. and Aldrich R. W., 2000. Vasoregulation by the beta1 subunit of the calcium-activated potassium channel. Nature 407, 870-6.
Catterall, W. A., 1995. Structure and function of voltage-gated ion channels. Annu Rev Biochem 64, 493-531.
Cui, J., Yang, H. and Lee, U.S., 2009. Molecular mechanisms of BK channel activation. Cell Mol Life Sci 66, 852-75.
Fettiplace, R. and Fuchs, P. A., 1999. Mechanisms of hair cell tuning. Annu Rev Physiol 61, 809-34.
Ghatta, S., Nimmagadda, D., Xu, X. and O'Rourke, S. T., 2006. Large-conductance, calcium-activated potassium channels: structural and functional implications. Pharmacol Ther 110, 103-16.
Gormemis, A. E., Ha, T. S., Im, I., Jung, K. Y., Lee, J. Y., Park, C. S. and Kim, Y. C., 2005. Benzofuroindole analogues as potent BK(Ca) channel openers. Chembiochem 6, 1745-8.
Ido, K., Ohwada, T., Yasutomi, E., Yoshinaga, T., Arai, T., Kato, M., Sawada, K., 2012 Screening quality for Ca(2+)-activated potassium channel in IonWorks Quattro is greatly improved by using BAPTA-AM and ionomycin. J Pharmacol Toxicol Methods http://dx.doi.org/10.1016/j.vascn.2012.10.005.
Jiang, Y., Pico, A., Cadene, M., Chait, B. T. and MacKinnon, R., 2001. Structure of the RCK domain from the E. coli K+ channel and demonstration of its presence in the human BK channel. Neuron 29, 593-601.
Kim, H. J., Lim, H. H., Rho, S. H., Bao, L., Lee, J. H., Cox, D. H., Kim do, H. and Park, C. S., 2008. Modulation of the conductance-voltage relationship of the BKCa channel by mutations at the putative flexible interface between two RCK domains. Biophys J 94, 446-56.
Lee, B. C., Lim, H. H., Kim, S., Youn, H. S., Lee, Y., Kim, Y. C., Eom, S. H., Lee, K. W. and Park, C. S., 2012. Localization of a site of action for benzofuroindole-induced potentiation of BKCa channels. Mol Pharm 82, 143-55.
Lee, U. S. and Cui, J., 2010. BK channel activation: structural and functional insights. Trends Neurosci 33, 415-23.
Meredith, A. L., Thorneloe, K. S., Werner, M. E., Nelson, M. T. and Aldrich, R. W., 2004. Overactive bladder and incontinence in the absence of the BK large conductance Ca2+-activated K+ channel. J Biol Chem. 279, 36746-52.
Nelson, M. T., Cheng, H., Rubart, M., Santana, L. F., Bonev, A. D., Knot, H. J. and Lederer, W. J., 1995. Relaxation of arterial smooth muscle by calcium sparks. Science 270, 633-7.
Salkoff, L., Butler, A., Ferreira, G., Santi, C. and Wei, A., 2006. High-conductance potassium channels of the SLO family. Nat Rev Neurosci 7, 921-31.
Werner, M. E., Zvara, P., Meredith, A. L., Aldrich, R. W. and Nelson, M. T., 2005. Erectile dysfunction in mice lacking the large-conductance calcium-activated potassium (BK) channel. J Physiol 567, 545-56.
Zheng, W., Spencer, R. H. and Kiss, L., 2004. High throughput assay technologies for ion channel drug discovery. Assay Drug Dev Technol. 2, 543-52.

1. A method for screening ion channel modulators, comprising:
(a) treating a cell comprising a nucleotide sequence encoding an amino acid sequence in which amino acids at positions G733 and N736 in a wild type BK_{Ca} channel gene are mutated with a test material; and
(b) analyzing the activity of the mutated BK_{Ca} channel in the cell, wherein the test material is determined to be an ion channel activator when the test material potentiates the activity of the mutated BK_{Ca} channel, and the test material is determined to be an ion channel inhibitor when the test material inhibits the activity of the mutated BK_{Ca} channel.

2. The method for screening according to claim 1, wherein the mutated amino acid sequence in step (a) is the amino acid sequence in which amino acids at positions G733 and N736 in the wild type BK_{Ca} channel gene are mutated to G733D and N736K.

3. The method for screening according to claim 1, wherein the mutated BK_{Ca} channel in step (b) is activated by membrane depolarization regardless of [Ca²⁺]ᵢ.

4. The method for screening according to claim 3, wherein the depolarization is induced stepwise ranging from −80 mV to 200 mV in increments of 10 mV.

5. The method for screening according to claim 1, wherein conductance-voltage relationships (G-V) for the mutated BK_{Ca} channel in step (b) shift in the negative voltage direction.

6. The method for screening according to claim 1, wherein the analysis in step (b) is performed through fluorescence measurement for T1⁻ ion concentration.

7-9. (canceled)

10. A method for screening a therapeutic agent for BK_{Ca} channel activity-associated diseases, disorders or conditions, comprising:
(a) treating a cell comprising a nucleotide sequence encoding an amino acid sequence in which amino acids at positions G733 and N736 in a wild type BK_{Ca} channel gene are mutated with a test material; and
(b) analyzing the activity of the mutated BK_{Ca} channel in the cell,
wherein the test material is determined to be a therapeutic agent for BK_{Ca} channel activity-associated diseases, disorders or conditions when the test material potentiates the activity of the mutated BK_{Ca} channel.

11. The method for screening according to claim 10, wherein the mutated amino acid sequence in step (a) is the amino acid sequence in which amino acids at positions G733 and N736 in the wild type BK_{Ca} channel gene are mutated to G733D and N736K.

12. The method for screening according to claim 10, wherein the BK_{Ca} channel activity-associated disease, disorder or condition is cardiovascular diseases, obstructive or inflammatory airway diseases, lower urinary tract disorders, erectile-dysfunction, anxiety and anxiety-related conditions, epilepsy or pain.

13. The method for screening according to claim 12, wherein the cardiovascular diseases are atherosclerosis, atherothrombosis, coronary artery disease, ischemia, reperfusion injury, hypertension, restenosis, arteritis, myocardial ischemia or ischemic heart diseases, stable and unstable angina, stroke, congestive heart failure, aortic disease, such as aortic coarctation or aortic aneurysm, or peripheral vascular disease.

14. The method for screening according to claim 12, wherein the obstructive or inflammatory airway diseases are hyperactive airway response, pneumoconiosis, aluminosis, anthracosis, asbestosis, lithosis, ptilosis, siderosis, silicosis, tobacco toxicosis, byssinosis, sarcoidosis, berylliosis, emphysema, acute respiratory distress syndrome (ARDS), acute lung injury (ALI), acute or chronic infectious pulmonary disease, chronic obstructive pulmonary disease (COPD), bronchitis, chronic bronchitis, wheezy bronchitis, hyperactive airway response or aggravated cystic fibrosis, or cough including chronic cough, aggravated hyperactive airway response, pulmonary fibrosis, pulmonary hypertension, inflammatory pulmonary disease, and acute or chronic respiratory infection disease.

15. The method for screening according to claim 12, wherein the lower urinary tract disorders are overactive bladder, unstable bladder, overactive detrusor muscle, detrusor instability, detrusor hyperreflexia, sensory urgency to urinate, urinary incontinence, urge urinary incontinence, urinary stress incontinence, reflex urinary incontinence, slow urination, terminal dribbling, dysuria, or spastic bladder.

16. The method for screening according to claim 12, wherein the anxiety and anxiety-related conditions are generalized anxiety disorder, panic disorder, obsessive compulsive disorder, social phobia, performance anxiety, posttraumatic stress disorder, acute stress response, adjustment disorder, hypochondria, separation anxiety disorder, agoraphobia, or specific phobias.


SEQUENCE LISTING (4 sequences)

SEQ ID NO: 1 - DNA, 3534 bp, Homo sapiens
atggcaaatggtggcggcggcggcggcggcagcagcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctt

SEQ ID NO: 2 - protein, 1178 aa, Homo sapiens
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 3 - DNA, 3339 bp, Artificial
  nucleotide sequence of mutated BKCa channel
atggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcgacctccggaaactggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctt

SEQ ID NO: 4 - protein, 1113 aa, Artificial
  amino acid sequence of mutated BKCa channel
MDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIDLRKLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL